(19) United States
(12) Patent Application Publication
(10) Pub. No.: US 2008/0118528 A1
(43) Pub. Date: May 22, 2008

(54) Synthesis and Purification of West Nile Virus Virus-Like Particles

(76) Inventors: Tsanyang Jake Liang, North Bethesda, MD (US); Ming Qiao, South Australia (AU); Ashok Mundrigi, Karnataka (IN); Walter I. Lipkin, New York, NY (US)

Correspondence Address: KNOBBE, MARTENS, OLSON & BEAR, LLP, 2040 MAIN STREET, FOURTEENTH FLOOR, IRVINE, CA, 92614, US

(21) Appl. No.: 11/579,459

(22) PCT Filed: May 2, 2005
(86) PCT No.: PCT/US2005/15319

§ 371 Date: Dec. 3, 2007

Related U.S. Application Data
(60) Provisional application No. 60/568,013, filed on May 4, 2004.

(51) Int. Cl. A61K 39/12; C12P 21/04; C07H 21/04; C12N 15/00; C12N 5/00; G01N 33/53

(52) U.S. Cl. 424/204.1; 435/69.3; 536/23.72; 435/320.1; 435/348; 435/7.1

(57) ABSTRACT

The present invention relates to virus-like particles derived from West Nile Virus and to methods for generating the same. These particles are useful in diagnostic applications, and as components of vaccines directed at preventing the incidence of disease.

### RELATED APPLICATIONS

This application claims the benefit of U.S. Provisional Patent Application No. 60/567,013, filed May 4, 2004, which is incorporated by reference herein in its entirety.

### FIELD OF THE INVENTION

The present invention relates to virus-like particles derived from West Nile Virus and to methods for generating the same. These particles are useful in diagnostic applications, and as components of vaccines directed at preventing the incidence of disease.

### BACKGROUND OF THE INVENTION

West Nile Virus (WNV) is a member of the Japanese encephalitis antigenic complex within the family Flaviviridae, genus Flavivirus (Calisher, C. H. 1988 Acta Virol 32:469-478; Heinz, F. X., and Allison, S. L. 2000 Adv Virus Res 55:231-269). Other pathogens within this complex include Alfuy, Cacipacore, Koutango, Japanese encephalitis (JEV), Murray Valley encephalitis (MVEV), St. Louis encephalitis (SLEV), Usutu, and Yaounde viruses (Heinz, F. X., and Allison, S. L. 2000 Adv Virus Res 55:231-269). WNV is primarily arthropod-borne; mosquitoes are the primary vector for transmission amongst vertebrate hosts. Outbreaks of human WNV infection have been reported throughout the Middle East, Sub-Saharan Africa, Europe, Asia, and, recently, North America (Asnis et al. 2000 Clin Infect Dis 30:413-418; Briese, T. et al. 1999 Lancet 354:1261-1262; Lanciotti, R. S., et al. 1999 Science 286:2333-2337; Anderson, J. F. et al. 1999 Science 286:2331-2333). Two genetic lineages are established based on signature motifs in envelope gene sequence (Berthet, F. X. et al. 1997 J Gen Virol 78:2293-2297). Whereas lineage I viruses are associated with outbreaks of acute human disease, lineage II viruses appear to be confined to endemic, enzootic cycles. Flaviviruses infect permissive cells by receptor-mediated endocytosis. Different patterns in neuroinvasiveness and neurovirulence of flaviviruses are typically associated with changes in the E protein sequence and occasionally with mutations in the nonstructural genes (McMinn, P. C. 1997 J Gen Virol 78:2711-2722; Monath, T. P., et al. 1983 Lab Invest 48:399-410); host genetic factors are also postulated. SLEV and MVEV have been reported to gain entry into the brain by migration along olfactory neurons (McMinn, P. C. 1997 J Gen Virol 78:2711-2722; Chambers, T. J., et al. 1998 J Gen Virol 79:2375-2380). It is postulated that these viruses and WNV infect the olfactory neuroepithelium via blood capillaries during the viremic phase.

Similar to the other members of the Japanese encephalitis virus (JE) serogroup, WNV infection typically causes subclinical or nonspecific mild febrile illnesses lasting 3-5 days. However, 1-2% of infections can progress to fatal neurological disease involving profound motor weakness and axonal neuropathy (Asnis, D. S. et al. 2000 Clin Infect Dis 30:413-418). A killed virus equine vaccine is in use (Tesh, R. B. and Arroyo J. 2002 Emerg Infect Dis 8:1392-1397); however, no human vaccine is approved. Although passive immunotherapy has been shown to be effective in mouse models (Ben-Nathan, D., et al. 2003 J Infect Dis 188:5-12; Engle, M. J. and Diamond, M. S. 2003 J Virol 77:12941-12949), its use has been limited in humans (Agrawal, A. G. and Petersen, L. R. 2003 J Infect Dis 188:1-4). Neither a treatment option nor a proven vaccine for the prevention of WNV infection is available at the present time.

### SEGUE TO THE INVENTION

There is an urgent need for an effective prophylactic vaccine to prevent West Nile Virus (WNV) transmission and infection in domestic animals and humans. Several approaches to the development of a WNV vaccine development have demonstrated immunogenicity and protective efficacy including the chimeric (Monath, T. P. 2001 Ann N Y Acad Sci 951:1-12; Pletnev, A. G. et al. 2002 PNAS USA 99:3036-3041), DNA (Davis, B. S., et al. 2001 J Virol 75:4040-4047; Hall, R. A. et al. 2003 PNAS USA 100:10460-10464), and live attenuated vaccines (Lustig, S., et al. 2000 Viral Immunol 13:401-410). Virus-like particles (VLPs) synthesized in various expression systems have been used to prevent infection with papillomaviruses (Koutsky, L. A. et al. 2002 N Eng J Med 347:1645-1651) and rotaviruses (Madore, H. P. et al. 1999 Vaccine 17:2461-2471). Such an approach has also been successfully extended to other important human pathogens such as flaviviruses (Konishi, E. et al. 1992 Virology 188:714-720; Kroeger, M. A. and McMinn, P. C. 2002 Arch Virol 147:1155-1172; Qiao, M. et al. 2003 Hepatology 37:52-59). In this disclosure, we report the production of WNV-like particles (WNV-LPs) containing the WNV structural proteins, prME and CprME, by use of a recombinant baculovirus in insect cells, and we evaluate the use of WNV-LPs as a vaccine.

### SUMMARY OF THE INVENTION

The present invention relates to virus-like particles derived from West Nile Virus and to methods for generating the same. These particles are useful in diagnostic applications, and as components of vaccines directed at preventing the incidence of a WNV-mediated disease.

### BRIEF DESCRIPTION OF THE DRAWINGS

FIG. 1. Translation and processing of the flavivirus protein. At the top is depicted the viral genome with the structural and nonstructural protein coding regions, the 5′ cap, and the 5′ and 3′ NCRs indicated. Boxes below the genome indicate precursors and mature proteins generated by the proteolytic processing cascade. Mature structural proteins are indicated by shaded boxes and the nonstructural proteins and structural protein precursors by open boxes. Contiguous stretches of uncharged amino acids are shown by black bars. Asterisks denote proteins with N-linked glycans but do not necessarily indicate the position or number of sites utilized. Cleavage sites for host signalase (♦), the viral serine protease (↓), furin or other Golgi-localized protease (♡), or unknown proteases (?) are indicated. Lindenbach, B. D. & Rice, C. M., “Flaviviridae: The Viruses and Their Replication,” in Fields Virology (Knipe, D. M. & Howley, P. M., eds., 4^{th} ed., 2001 Lippincott Williams & Wilkins).

FIG. 2. Construction and production of West Nile virus-like particles (WNV-LPs) in insect cells. A. Map depicting segments of the WNV genome in the recombinant baculovirus expression vector; the bvWNVprME construct (top) contains the coding sequences for prM and E and the bvWNVCprME construct (bottom) contains the coding sequences for core, prM and E. pPolh, baculovirus polyhedrin promoter; SV40pA, simian virus 40 polyadenylation sequence. B. Characterization of WNV-LPs. WNV-LPs were purified from Sf9 cells by iodixanol gradient centrifugation. Ten fractions collected from the top of the gradient were analyzed for total protein content and the titer of WNV E protein by ELISA. C. Western blot analysis of purified prME-like particles (prME-LPs) and CprME-like particles (CprME-LPs) with rabbit anti-E or -M antibodies. Uninfected Vero cells and hepatitis C virus-like particles (HCV-LPs) were used as negative controls and WNV-infected Vero cells were used as a positive control. D. Cryoelectron micrograph (CM) of purified prME-LPs. Bar, 100 nm.

FIG. 3. Detection of virus and viral RNA in serum, spleen and brain in immunized mice after challenge with WNV. Mice were bled 3 days after challenge for determination of viremia by either plaque forming assay (PFU/ml) or real-time RT-PCR (copies/ml). Spleens and brains were harvested from mice at death or 31 days after challenge. RNA was extracted and analyzed by real-time RT-PCR. The results of individual mice are shown. The Y-axis scale is set up to start with value near the cut-off of the assay.

### DETAILED DESCRIPTION OF THE PREFERRED EMBODIMENT

No specific vaccine for West Nile Virus (WNV) is currently available for human use. In this disclosure, we describe the generation of WNV-like particles (WNV-LPs) in insect cells by use of recombinant baculoviruses expressing the WNV structural proteins prME or CprME. BALB/c mice immunized with purified WNV-LPs developed WNV-specific antibodies that had potent neutralizing activities. Mice immunized with prME-like particles (prME-LPs) showed no morbidity or mortality after challenge with WNV. Immunization with prME-LPs can induce sterilizing immunity without producing any evidence of viremia or viral RNA in the spleen or brain. Based on these results, WNV-LPs are envisioned as a vaccine for the control of WNV infection.

### West Nile Virus Virus-like Particles

According to the invention there is provided a method for the production of virus-like particles (VLPs) from a West Nile Virus (WNV), said method comprising the steps of:

a) expressing a construct comprising the prM and E genes of a WNV in a baculoviral expression cassette and cloned under the control of a promoter in insect cells;

b) culturing the insect cells for a sufficient period of time to allow production of baculovirus particles; and

c) separating the VLPs from the baculoviral particles and the insect cells.

The virus-like particles (VLPs) produced according to the invention have thus been generated using the baculovirus expression system. These VLPs are suitable for use as diagnostic antigens, particularly in methods such as enzyme linked immunosorbent assay (ELISA) and in lateral-flow rapid test-type kits. The VLPs may also be used in vaccines.

The inventors have discovered that the baculovirus expression system is advantageous for the production of WNV VLPs. This expression system has been used previously for the production of various other virus VLPs. However, to date, it was not appreciated that this expression system would be useful in the expression of a WNV prM/E cassette or in VLP production.

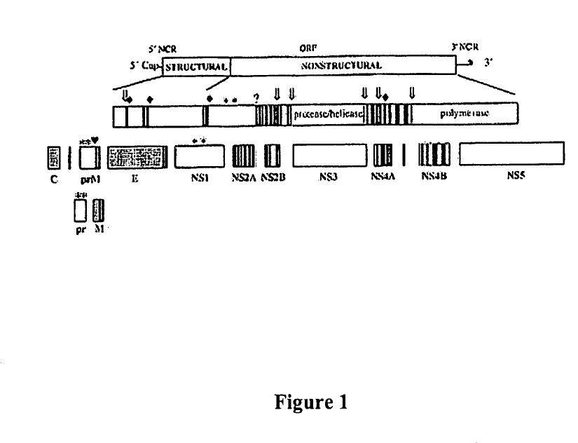

The RNA genome of a flavivirus is enclosed by a capsid or core (C) protein that is surrounded by a host-derived lipid membrane containing the glycosylated viral membrane (M) and envelope (E) proteins (FIG. 1). Seven non-structural proteins (NS1, NS2a, NS2b, NS3, NS4a, NS4b, NS5) provide the replicative and proteolytic functions for virus replication.

The E protein is the dominant antigenic determinant of humoral and cellular immune responses in WNV infections. Earlier attempts at cloning the E gene showed that they did not necessarily exhibit the same activity or conformation as the native protein. It is now believed that correct folding of the flavivirus E protein also requires the co-ordinated synthesis of prM protein.

Wild-type virion assembly occurs first by proteolytic cleavage of the polyprotein at the M/E cleavage site by a cellular signalase found in the endoplasmic reticulum, to form immature virions composed of heterodimeric prM and E proteins. prM in turn is cleaved by a cellular protease (furin) in acidic particles of the trans-Golgi network that leads to the release of mature particles. In some embodiments, the invention contemplates the addition of a signalase cleavage site located in the prM gene to mediate the cleavage of prM to form the structural protein M. In other embodiments, the invention contemplates the addition of a furin cleavage site located at the junction of the prM/E genes to mediate the cleavage of the polyprotein (WO 03/062408 published 31 Jul. 2003).

According to the method of the invention, the VLPs are derived from the West Nile Virus. The applicability of this method to produce VLPs in insect cells from all members of the WNV taxonomic group is inferred by the observation that the properties of other WNV strains is similar to that of any one WNV strain. (Brinton, M. A. 2002 Annu. Rev. Microbiol. 56:371-402.) WNV isolates have been grouped into two genetic lineages (1 and 2) on the basis of signature amino acid substitutions or deletions in their envelope proteins. All the WNV isolates associated thus far with outbreaks of human disease have been in lineage 1. Lineage 2 viruses are restricted to endemic enzootic infections in Africa.

The prM and E genes from a West Nile Virus are known in the art. Accordingly, the skilled reader, imbued with the present teaching, will be capable of practicing the invention for a West Nile Virus without the need for inventive skill. Sequences of the relevant genes from a West Nile Virus may be found in publicly-available databases such as GenBank (ncbi.nlm.nih.gov), EMBL (ebi.ac.uk) and DDBJ (ddbj.nig.ac.jp).

Preferably, the entire prM and E genes are used, although fragments of these proteins may be used, provided that intact VLPs are still generated. Furthermore, alterations from the wild type sequence may be allowed in the sequences of the prM and E genes, including insertions and deletions, particularly if the insertions or deletions only involve a few amino acids, e.g., under thirty, and preferably under ten, and do not remove or displace amino acids that are critical to a functional conformation, e.g., cysteine residues. Substitutions, particularly conservative amino acid substitutions, may also be allowed in the sequences of the proteins. Such altered prM and E genes are referred to herein as “variants” of the wild type proteins.

It may also be preferable to incorporate sequence from neighboring genes in the sequence of the viral polyprotein, such as the capsid (C) gene that abuts the prM gene, and the NS1 gene that abuts the E gene.

In order to practice the method of the present invention, it will be necessary to generate a baculovirus for infection of an insect cell that includes a construct comprising the prM and E genes of a WNV, or a variant of the prM and/or E genes expressed under the control of a promoter in a baculoviral expression cassette. The step of generating a baculovirus and its infected insect cell may form a part of the method of the present invention.

Methods for the generation of such constructs will be apparent to those of skill in the art from reading the present specification and using teachings published in the literature. For example, methods and materials for baculovirus/insect cell expression systems are commercially available in kit form from, inter alia, Gibco-Invitrogen Corporation, Carlsbad, Calif. (the Bac-to Bac system). These techniques are generally known to those skilled in the art and are described fully in Summers and Smith, Texas Agricultural Experiment Station Bulletin No. 1555 (1987). In brief, in the classical system, linearized baculovirus (Autographa californica) DNA containing the LacZ gene is co-transfected into Spodoptera frugiperda (Sf) insect cells with the shuttle plasmid containing the insert, flanked by polyhedrin promoter sequences. Homologous recombination replaces the LacZ gene with the insert to produce viable viruses. Plaques are screened for the insert by blue/white selection using X-gal as a substrate in plaque assays.

The Bac-to-Bac system is more efficient than the classical system because recombination occurs in bacteria already containing baculoviral DNA (bacmid). This means that the multiple rounds of plaque purification required by the classical system are no longer necessary, so that expression is significantly faster using this system. The Bac-to-Bac system and similar systems that share these advantageous features are thus preferred according to the methods of the present invention.

All these systems use a baculoviral expression cassette that includes a polyhedrin promoter. By “baculoviral expression cassette” is meant a portion of nucleic acid that contains regulatory signals necessary for the transcription of the proteins(s) encoded by genes whose transcription is controlled by the regulatory signals in the cassette. It is, however, not essential for the working of the present invention that the polyhedrin promoter is used. Any “late promoter” that is effective to drive transcription in insect cells may be used. Preferably, very late promoters such as the polyhedrin and p10 promoters are used. A polyhedrin promoter may be defined as the 5′ noncoding region of the polyhedrin gene. Proteins under the control of the very late promoters, such as p10 and polyhedrin, can account for up to 50% of the total cell mass during baculovirus infection. Foreign gene inserts under control of the polyhedrin promoter can produce high levels of recombinant protein expression.

Particularly suitable host cells for use in this system include insect cells such as Spodoptera Sf9 cells (Invitrogen). High5 (Tn5) cells (Invitrogen) are also envisioned. High5 cells are suitable for use in the method of the invention, since these cells have been found to lead to higher expression levels of intact, immunogenic VLPs.

According to the method of the invention, insect cells in which the VLPs of the invention are expressed should be cultured for a sufficient period of time to allow production of baculovirus particles. This period of time will vary according to the particular system used and, potentially, the particular WNV from which the genes used in the system are derived. This period of time will be apparent to those of skill in the art. If in any doubt, the optimum period of time may be found by incubating the inset cells under various conditions and analyzing the quantity and quality of VLPs that are generated. Generally, the culture time will vary between 1 and 10 days and will optimally be around 5 days.

After a sufficient period of culturing, the VLPs must be separated from the baculoviral particles and the insect cells in order to allow their subsequent use, such as in the diagnostic and vaccine applications discussed below. Any method may be used that allows the efficient separation of VLPs from baculoviral particles and insect cells. Centrifugation is one preferred method. In one embodiment, the insect cells are pelleted by centrifugation, and the VLPs are harvested by lysis of the resulting cell pellet. In another embodiment, the insect cells are pelleted by centrifugation, and the VLPs are harvested from the resulting supernatant (WO 03/062408 published 31 Jul. 2003). If necessary, VLPs may be further purified, for example, using a sucrose, CsCl, or other type of equilibrium gradient centrifugation in accordance with standard methods known to those of skill in the art.

According to a further aspect of the invention, there is provided a composition of matter comprised of VLPs obtained according to any one of the methods of the invention described above. The invention also provides pharmaceutical compositions comprising a preparation of such VLPs, in combination with a suitable pharmaceutical carrier. A thorough discussion of pharmaceutically acceptable carriers is available in Remington's Pharmaceutical Sciences (Mack Pub. Co., N.J. 1991).

VLPs produced according to the methods of the present invention have a large number of applications, including as therapeutic or diagnostic reagents, as vaccines, or as other immunogenic compositions. Particularly preferred applications lie in the fields of diagnosis and vaccination.

Accordingly, a further aspect of the invention provides for the use of a composition according to the above-described aspect of the invention in diagnosis of a WNV-mediated disease or in a method of diagnosis. For example, VLPs generated according to the present invention may form a component of a diagnostic kit.

Such assays may also be used to evaluate the efficacy of a particular therapeutic treatment regimen in animal studies, in clinical trials or in monitoring the treatment of an individual patient, or in epidemiological studies.

A number of methods exist for the diagnosis of disease that utilize recombinant preparations of protein. Such assays generally detect antibody specific for WNV proteins that circulates in patient sera and include methods that utilize recombinant VLPs and a label to detect circulating antibody in human body fluids or in extracts of cells or tissues. All appropriate methodologies may utilize the recombinant VLPs generated by a method according to the present invention.

Assay techniques that can be used to determine levels of a polypeptide of the present invention in a sample derived from a host are well-known to those of skill in the art and include membrane, solution, or chip based technologies for the detection and/or quantification of antibody (particularly IgG and IgM) (see Hampton, R. et al. (1990) Serological Methods, a Laboratory Manual, APS Press, St Paul, Minn.; and Maddox, D. E. et al. 1983 J. Exp. Med. 158:1211-1216). Examples include techniques such as radioimmunoassays (RIA), competitive-binding assays, Western Blot analysis, ELISA (such as direct and capture techniques) and FACS assays and include membrane, solution, or chip based technologies for the detection and/or quantification of antibody (see Hampton, R. et al. (1990) Serological Methods, a Laboratory Manual, APS Press, St Paul, Minn.; and Maddox, D. E. et al. 1983 J. Exp. Med. 158:1211-1216).

This aspect of the invention thus provides a diagnostic method that comprises the steps of: (a) contacting a VLP preparation as described above with a biological sample under conditions suitable for the formation of a polypeptide-antibody complex; and (b) detecting said complex. In these techniques, body fluids or cell extracts taken from a patient are contacted with recombinant VLPs under conditions suitable for coinplex formation. Complex will only form with antibodies if antibodies are present in the body fluid or cell extract. The amount of standard complex formation may be quantified by various methods, such as by photometric means. Inclusion of appropriate controls ensures the credibility of such systems.

Samples for diagnosis may be obtained from a patient subject's cells or bodily fluids, such as from blood, urine, saliva, tissue biopsy or autopsy material.

VLPs may be used either with or without modification, and may be labeled by joining them, either covalently or non-covalently, with a reporter molecule to aid detection of complex. A wide variety of reporter molecules known in the art may be used. Examples include suitable radionuclides, enzymes and fluorescent, chemiluminescent or chromogenic agents as well as substrates, cofactors, inhibitors, magnetic particles, and the like. When unlabelled VLPs are used, in order to detect the presence of antibody molecules in patients, it may be preferable to use labels that are specific for patient antibodies.

A preferred diagnostic method is an ELISA-based method.

According to a still further aspect of the invention, there is provided a diagnostic kit comprising a preparation of recombinant VLPs generated according to any one of the methods of the invention described above. Preferably, such a diagnostic kit will additionally incorporate at least one reagent useful for the detection of a binding reaction between the antibody and the polypeptide. Such kits will be of use in diagnosing a WNV-mediated disease. As the skilled reader will understand, a number of different preparations of VLPs, appropriately labeled to allow their respective distinction may be used, in order to detect the presence of WNV-specific antibodies of different types. Furthermore, the VLPs of the invention may be used in conjunction with one or more other systems as a combined diagnostic system for the detection of a range of different disorders and/or diseases.

The VLPs of the invention may also be used as components of vaccines. Accordingly, this aspect of the invention includes the use of a composition according to the above-described aspect of the invention in a vaccine or in a method of vaccination. In this aspect of the invention, the VLPs are used to raise antibodies against the disease-causing agent (WNV).

Vaccines according to this aspect of the invention may either be prophylactic (i.e. to prevent infection) or therapeutic (i.e. to treat disease after infection). Such vaccines will comprise the immunizing VLPs, usually in combination with a pharmaceutically-acceptable carrier as described above, which include any carrier that does not itself induce the production of antibodies harmful to the individual receiving the composition. Additionally, these carriers may function as immunostimulating agents (“adjuvants”). Furthermore, the antigen or immunogen may be conjugated to a bacterial toxoid, such as a toxoid from diphtheria, tetanus, cholera, H. pylori, or from other pathogens.

Adjuvants include but are not limited to QS-21, CpG, MPL, Titer Max, MoGM-CSF, CRL-1005, PF-026, GPI-0100, GM-CSF and combinations thereof (Kim et al., 2000 Vaccine 19:530-537).

Since polypeptides such as VLPs may be broken down in the stomach, vaccines are preferably administered parenterally (for instance, by subcutaneous, intramuscular, intravenous, intrathecal or intradermal injection). Formulations suitable for parenteral administration include aqueous and non-aqueous sterile injection solutions that may contain antioxidants, buffers, bacteriostats and solutes that render the formulation isotonic with the blood of the recipient, and aqueous and non-aqueous sterile suspensions that may include suspending agents or thickening agents.

The vaccine formulations of the invention may be presented in unit-dose or multi-dose containers. For example, sealed ampoules and vials may be stored in a freeze-dried condition requiring only the addition of the sterile liquid carrier immediately prior to use. The dosage will depend on the specific activity of the vaccine and can be readily determined by routine experimentation. Furthermore, a number of different VLP preparations according to the invention may be administered as a combination vaccine, for example, to target a combination of different flavivirus-mediated diseases. Additionally, vaccine components specific for unrelated disorders might be included in a combination vaccine, for reasons' of program management, enhanced efficacy or, more usually, for lowered cost of administration and preparation.

According to a further aspect of the invention, there is provided a nucleotide construct for use in any one of the aspects of the invention described above. Such a nucleotide construct comprises the prM and E genes of a WNV, or a variant of the WNV and/or E genes, cloned under the control of a promoter in a baculoviral expression cassette. As discussed above, the promoter used in the construct is preferably a polyhedrin promoter or a p10 promoter. The invention also provides a vector comprising such a nucleotide construct and insect host cells comprising such a nucleotide construct or vector.

### Induction of Sterilizing Immunity against West Nile Virus by Immunization with West Nile Virus-Like Particles Produced in Insect Cells

### Production of WNV-Like Particles in Insect Cells.

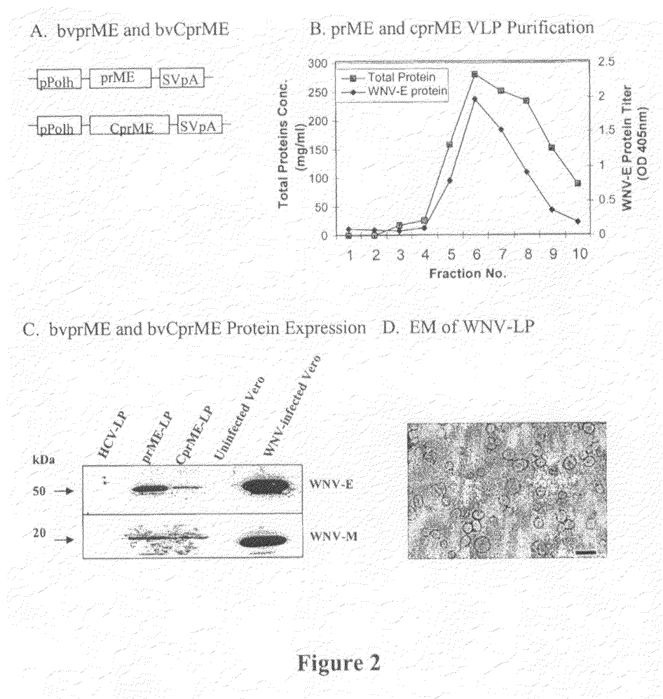

Recombinant baculoviruses bvWNVprME and bvWNVCprME (FIG. 2A) which contain the coding sequences for prM and E and for core, prM, and E, respectively, were shown to direct the production of WNV-LPs in insect cells. By use of a modified method described elsewhere for HCV-LPs (Jeong, S. H. et al. 2004 J Virol 78:6995-7003), WNV-LPs were harvested from bvWNVprME or bvWNVCprME-infected Sf9 cells by gentle permeabilization of cells with 0.5% digitonin and then purified by iodixanol gradient centrifugation. WNV E protein was detected by ELISA using galanthus nivalis lectin-coated microtiter plate (FIG. 2B). The peak of E reactivity corresponds to the peak total protein concentration and to buoyant densities of 1.12-1.14 g/ml. Western blot analysis (FIG. 2C) revealed that these fractions contain a 50-kDa E protein band and a 20-kDa prM band in both the prME-LP and CprME-LP preparations. The mature form of M protein was not detected, probably because the furin that is required for the proper cleavage of prM to M is not expressed efficiently in Sf9 insect cells (Yamshchikov, G. V. et al. 1995 Virology 214:50-58). A core protein band was also detected at 12 kDa in the CprME-LP preparation. Examination by cryoelectron microscopy revealed that WNV-LPs are polymorphic in appearance and have a diameter of 40-60 nm (FIG. 2D). The typical yield of WNV-LPs from the procedure is ˜1-2 mg/100 ml of culture, which is substantially greater than the reported yields of other flavivirus-like particles generated in mammalian cells (Konishi, E. and Fujii, A. 2002 Vaccine 20:1058-1067; Kojima, A. et al. 2003 J Virol 77:8745-8755).

### Induction of Neutralizing Antibodies to WNV.

Groups of BALB/c mice (n=6) were immunized with prME-LPs alone, CprME-LPs alone, prME-LPs plus AS01B, or AS01B alone, with 4 injections given at 3-week intervals. Mice were bled prior to and 2 weeks after each injection. Although all of the mice immunized with prME-LPs (with or without the AS01B adjuvant) developed anti-E antibodies after the fourth immunization, AS01B enhanced the anti-E antibody response significantly, from 317 to 8128, and also enhanced the anti-M antibody response, from 50 to 142 (table 1). CprME-LPs induced weaker antibody responses to the M and E proteins. One mouse in the AS01B group died of unknown causes after the first immunization.

TABLE 1
Antibody response in mice immunized with West Nile virus-like particles (WNV-LPs)

| | ELISA | | | | | Neutralization | |
|---|---|---|---|---|---|---|---|
| | Anti-WNV E protein | | anti-WNV M protein | | | Assay | |
| | Before | After | Before | After | NS1 | Before | After |
| Mouse Group | Challenge | Challenge | Challenge | Challenge | Seroconversion | Challenge | Challenge |
| Unimmunized/ | ND | <50 | ND | <50 | 0/6 | ND | 0 |
| unchallenged | | | | | | | |
| Unimmunized/ | ND | 2,432 | ND | <50 | 4/5 | ND | 15 |
| unchallenged | | | | | | | |
| AS01B | <50 | 3,200 | <50 | <50 | 4/4 | 0 | 37 |
| prME-LPs | 317 | 5,689 | <50 | 89 | 3/6 | 37 | 62 |
| prME-LPs | 8,128 | 45,709 | 112 | 355 | 1/6 | 75 | 75 |
| plusAS01B | | | | | | | |
| CprME-LPs | 86 | 7,217 | <50 | <50 | 5/6 | 0 | 57 |

Serum antibody titers were determined after the last of 4 immunizations. For each group, the geometric mean of the antibody titers was calculated. The titer for a mouse with a negative ELISA value at serum dilution of 50 was arbitrarily set at 50 for the calculation of geometric mean.
The results of statistical analyses were as follows (by Mann-Whitney U test or Fisher's exact test). Anti WNV E titer before challenge: P = 0.018, for prME-like particles (prME-LPS) vs. AS01B; P = 0.0009, for prME-LPs plus AS01B vs. AS01B; P = 0.026, for prME -LPs vs. prME-LPs plus AS01B. NS1 seroconversion: P = 0.024, for prME-LPs vs. AS01B, and P = 0.001, for prME-LPS plus AS01B vs. AS01B. Neutralizalion titer before challenge: P = 0.0007, for prME-LPsvs. AS01B, P = 0.0003, for prME-LPs plus AS01B vs. AS01B. CprME-LPs, CprME-like particles; ND, not done.

The pooled serum samples collected from each group at 2 weeks after the fourth immunization were assayed for titers of neutralizing antibodies (Table 1). Titers were determined to be 37 in the prME-LP group and 75 in the prME-LP plus AS01B group. The CprME-LP group did not develop detectable titers of neutralizing antibody. None of the serum samples from the AS01B group had any detectable antibodies to E and M proteins or neutralizing antibodies to WNV.

### Immunization with WNV-LP Protects Mice Against WNV Challenge.

Immunized mice were challenged with 10⁴ pfu of WNV. This dose is >100 times the ID₅₀ identified in a previous study in 6-month old BALB/c mice, and it was chosen to enhance the probability of discriminating differences in morbidity among groups. Mice were challenged 2 months after the 4th immunization (Table 2). Two groups of unimmunized mice (6 mice each) of similar age were included as control mice in this challenge experiment. One group was challenged with the same dose of WNV as were the immunized groups, and the other group was not challenged. Morbidity and mortality in the unimmunized/challenged group were 50% and 17%, respectively. There was no mortality or morbidity in either the prME-LP group or the prME-LP plus AS01B group. In contrast, 67% morbidity was observed in the CprME-LP group. The presence of high-titers of anti-E antibodies before challenge correlated with protective immunity, and all mice had a further increase in titers of anti-E antibodies after challenge, a result consistent with the presence of an anamnestic response directed towards the VLPs, of which the E protein is the major immunogenic component. All of the surviving mice were examined for pathologic abnormalities in the brain at the time of killing on day 31 after challenge. Hematoxylin/eosin (HE)-stained brain sections showed no significant neuropathologic damage.

TABLE 2
Protection of mice immunized with West Nile virus-like
particles (WNV-LPs) from challenge with WNV

| | | | | WNV detected in | | | |
|---|---|---|---|---|---|---|---|
| Mouse Group | Virus | Morbidity | Mortality | serum^{a} | serum^{b} | Spleen^{c} | Brain^{c} |
| Unimmunized/ | Mock | 0/6 | 0/6 | 0/6 | 0/6 | 0/6 | 0/6 |
| unchallenged | (Diluent) | | | | | | |
| Unimmunized/ | WNV | 3/6 | 1/6 | 5/6 | 6/6 | 6/6 | 3/6 |
| challenged | | | | | | | |
| AS01B | WNV | 2/5 | 1/5 | 4/5 | 4/5 | 5/5 | 2/5 |
| prME-LPs | WNV | 0/6 | 0/6 | 2/6 | 4/6 | 2/6 | 0/6 |
| prME-LPs plus + | WNV | 0/6 | 0/6 | 0/6 | 0/6 | 0/6 | 0/6 |
| AS01B | | | | | | | |
| CprME-LPs | WNV | 4/6 | 0/6 | 5/6 | 5/6 | 5/6 | 3/6 |

Data are proportion of mice. Two months after the last of 4 immunizations, mice were challenged intraperitoneally with 10⁴ pfu of WNV. The results of statistical analyses are as follows (by Fisher's exact test; control combines the results from the unimmunized/challenged and AS01B groups). Morbidity: P = 0.03, for prME-like particles (prME-LPS) vs. control, and P = 0.03, for prME-LPs plus AS01B vs. control. WNV detected in serum by plaque-forming assay: P = 0.04,for prME-LPs vs. control, and P = 0.0016, for prME-LPs plus AS01B vs. control. WNV detected in serum by reverse-transcription polymerase chain reaction (RT-PCR): P = 0.0002, for prME-LPs plus AS01B vs. control, and P = 0.01, for prME-LPs vs prME-LPS plus AS01B. WNV detected in spleen: P = 0.007 for prME-LPs vs. control, and P = 0.0005 for prME-LPs plus AS01B vs. control. WNV detected in brain: P = 0.003, for prME-LPs vs. control, and P = 0.003,for prME-LPs plus AS01B vs. control. CprME-LPs, CprME-like particles.
^{a}Positive for infectious WNV by plaque-forming assay on day 3 after challenge.
^{b}Positive for viral RNA by RT-PCR on day 3 after challenge.
^{c}Positive for viral RNA on day of death or killing.

### Sterilizing Immunity is Achieved by WNV-LP Immunization.

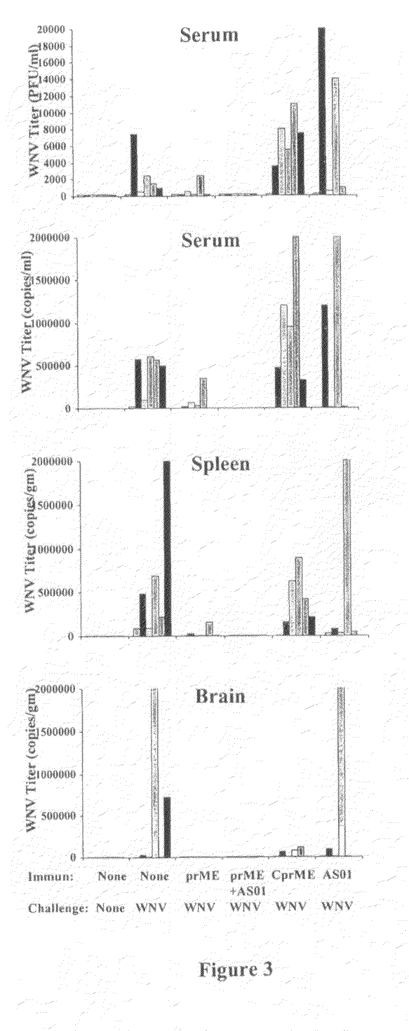

Viral replication was analyzed after challenge, to determine whether immunization with WNV-LPs induced sterilizing protective immunity. Viremia was assayed during the peak viremic phase on day 3 after challenge (Table 2 and FIG. 3). Because it is possible that the immunized mice had neutralizing antibodies by day 3, viremia was measured by both plaque-forming assay and RT-PCR. Postchallenge viremia (infectious virus or viral RNA) was detected in all 6 mice in the unimmunized/challenged group in 5 (83%) of the six mice in the CprME-LP group, and in 4 (67%) of the 6 mice in the prME-LP group; however, 0 of the 6 mice in the prME-LP plus AS01B group had circulating infectious virus or viral RNA in serum after challenge. Although 4 of the 6 mice in the prME-LP group had viral nucleic acid (as detected by RT-PCR), only 2 had infectious virus (as detected by plaque forming assay). In addition, the geometric mean viral titer of the prME-LP group (1.58×10⁴ copies/ml) was more than an order of magnitude lower than that of the unimmunized/challenged group (2×10⁵ copies/ml)(P=0.027).

As an additional measure of postchallenge viral replication, the presence of viral RNA in the spleen and brain was determined at time of death or at killing (day 31 after challenge). Viral RNA was detected in the brains of ˜50% of the mice in the unimmunized/challenged, AS01B, and CprME-LP groups (Table 2). In contrast, none of the mice that received either prME-LPs alone or prME-LPs plus AS01B bad detectable viral RNA in the brain, indicating that these mice were protected from neuroinvasion. Viral RNA was detected in the spleens of all the mice in the unimmunized/challenged and AS01B groups, providing evidence for active replication in these control groups. Viral RNA was detected in 2 of the 6 and 5 of the 6 mice in the prME-LP and CprME-LP groups, respectively, but in 0 of the mice in the prME-LP plus AS01B group. Thus viral replication was partially inhibited in the mice immunized with prME-LPs alone and was completely inhibited in the mice immunized with prME-LP plus AS01B.

Seroconversion to the WNV nonstructural protein NS1 was assayed after viral challenge. Eight of the 9 mice in the unimmunized/challenged and AS01B groups and 5 of the 6 mice in the CprME-LP group developed an anti-NS1 antibody response after challenge with WNV (Table 2). In contrast, only 3 of the 6 mice in the prME-LP group and 1 of the 6 mice in prME-LP plus AS01B group seroconverted to anti-NS1 antibody, indicating that immunization with prME-LPs (especially in the presence of adjuvant) prevented productive infection and therefore, exposure to NS1 after challenge with WNV. These results, together with the lack of detectable viremia and viral RNA in the spleens, indicate that sterilizing immunity occurred in mice immunized with prME-LPs.

It is not apparent why the CprME particles, differing from the prME particles only in the addition of core protein, are less immunogenic. One explanation could be that the CprME preparation is less pure, resulting in lower immunogenicity. It is also possible that the particles formed by the CprME construct are less immunogenic because of the subtle structural difference. Alternatively, the core protein may somehow diminish the immune response toward the VLPs.

It is interesting to note that the neutralization titer in the mice immunized with prME-LPs plus AS01B did not increase after challenge, probably because the preexisting neutralization titer was sufficient to protect the mice from infection. It is conceivable that cell-mediated immunity induced by immunization with VLPs might contribute to the observed sterilizing immunity (Qiao, M. et al. 2003 Hepatology 37:52-59). The relative contribution of humoral versus cellular components in the protective immunity observed here awaits future study.

Several published studies have described promising approaches to vaccine development for WNV. Chimeric or attenuated flaviviruses that are closely related to WNV have been shown to successfully protect animals from WNV infection (Monath, T. P. 2001 Ann N Y Acad Sci 951:1-12; Pletnev, A. G. et al. 2002 PNAS USA 99:3036-3041; Lustig, S. et al. 2000 Viral Immunol 13:401-410). DNA immunization by use of plasmid expressing WNV proteins (Davis, B. S. et al. 2001 J Virol 75:4040-4047) and Kunjin virus (Hall, R. A. et al. 2003 PNAS USA 100:10460-10464) have also been applied successfully in the animal model. Despite the promise of these vaccine candidates, safety concerns will always be an issue. However, VLP-based vaccines are noninfectious and are easily controlled for quality and safety. The recent successful development of a human papillomavirus vaccine based on VLP technology (Koutsky, L. A. et al. 2002 N Engl J Med 347:1645-1651) lends credence to the success of this approach in the development of an effective WNV vaccine.

### EXAMPLE 1

### Recombinant Baculovirus Constructs

Recombinant baculovirus expressing WNV prME and CprME was generated by use of the Bac-to-Bac baculovirus expression system (Invitrogen) as described elsewhere (Jeong, S. H. et al. 2004 J Virol 78:6995-7003). cDNA (GenBank accession number AF202541) for prME (nt 335-2427) and CprME (nt 1-2636) was generated from WNV strain HNY1999-infected Vero cells by polymerase chase reaction (PCR) with the following 2 primer sets: for prME, (5′ CTA TCA ATC GGC GGA GCT C3′) (SEQ ID NO: 1) and (5′ ACC CAG TGT CAG CGT GCA 3′) (SEQ ID NO: 2), and for CprME, (5′ GCG GGA TCC TAA TAC GAC TCA CTA TAG GGA GTA GTT CGC CTG TGT GAG CTG 3′) (SEQ ID NO: 3) and (5′ GC TTC CCA CAT TTG RTG YTC 3′) (SEQ ID NO: 4). These PCR generated fragments were then cloned into the pGEM-T Easy vector (Promega). pFASTBac-prME and pFASTBac-CprME were generated by subcloning an EcoRI and SpeI fragment into the pFASTBac-1 vector (Invitrogen). The correct recombinant baculoviruses were identified by immunofluorescence and immunoblotting with a rabbit anti-E antibody. Baculoviruses were amplified by additional rounds of Sf-9 cell infection until a final titer of 5×10⁷ plaque forming units (PFU)/ml was achieved.

### Production of WNV-LP

The procedure for the production and purification of WNV-LPs was similar to that for hepatitis C virus-like particles (HCV-LPs) (Jeong, S. H et al. 2004 J Virol 78:6995-7003), with some modifications. Briefly, Sf9 cells (2×10⁹) were infected with recombinant baculovirus at a multiplicity of infection (moi) of 5 to 10 and incubated at 27° C. for 3 days in Sf-900 serum-free medium (Gibco-Invitrogen Corporation, Carlsbad, Calif.). Cells were harvested by centrifugation at 2500×g for 5 min at room temperature and the cell pellet washed once with phosphate-buffered saline (PBS). The cell pellet was resuspended in 18 ml of pre-warmed PBS and 3 ml of 90% glycerol containing 10 mM HEPES buffer, 1 mM PMSF (Phenylmethysulfonyl Fluoride, Sigma-Aldrich, St. Louis, Mo.) and a cocktail of EDTA-free protease inhibitors (PI) (Roche, Indianapolis, Ind.). The cell suspension was mixed and incubated at 37° C. for 5 min. The process was repeated twice so that the final glycerol concentration in the cell suspension reached 30%. The cells were then chilled on ice for 5 min, and centrifuged at 2500×g for 10 min at 4° C. The following purification steps were performed at 4° C. unless specified. The cell pellet was resuspended with 50 ml lysis buffer (10 mM Tris-HCl [pH 7.4], 1 mM MgCl₂, 1 mM CaCl₂, 1 mM PMSF, PI) containing 0.5% digitonin and allowed to sit on ice with gentle agitation for 4 h. The cell lysate was centrifuged at 26,000×g in SW28 rotor (Beckman) for 30 min to remove cell debris. To maximize the WNV-LP yield, the lysis process may be repeated once more by resuspending the cell pellet in fresh lysis buffer containing 0.5% digitonin. The supernatant was loaded onto a 1.5 ml of 40% (wt/vol) iodixanol (Optiprep; Greiner Bio-one, Longwood, Fla.) cushion in TNC/PI buffer and centrifuged at 52,000×g for 6 h using SW41 rotor (Beckman) (Pietschmann, T. et al. 2002 J Virol 76:4008-4021). The supernatant was discarded, the interface and cushion (˜1.7 ml) were transferred into a fresh centrifuge tube (SW41) and overlaid with a linear iodixanol gradient (0-30%) and centrifuged at 110,000× g for 16 h in SW41 rotor. One-milliliter fractions were collected from the top of the tube, and the protein concentrations of fractions were determined by Coomassie Plus protein assay reagent (Pierce, Ill.) and E and M proteins by enzyme-linked immunosorbent assay (ELISA, see below) and Western blot. WNV-LP were analyzed by electron microscopy. UV-LP was prepared in large batches and stored at 4° C. until use. The WNV recombinant proteins prM, E, and NS1 were produced, and rabbit antibodies against them were generated, as described below.

### Expression of Recombinant WNV Proteins and Production of Anti-WNV Rabbit Antisera.

The prM (nt424-924), E (nt925-2427) and NS1 sequences (nt 2428-3483) were amplified from HNY1999 WNV strain and cloned into the vector pENTR1A (Invitrogen). After recombination into the vector pDEST17 and transformation in BL21pLYS cells, expression was induced with isopropyl-beta-D-thiogalactopyranoside. Following extraction under denaturing conditions with 8M urea, recombinant proteins were purified by nickel agarose chromatography, dialyzed and quantitated by Bradford assay (Bio-Rad). Purified proteins was submitted to Lampire Biologicals (Pipersville, Pa.) for generation of rabbit antisera to WNV prM, E, and NS1. Specificity of rabbit antisera was tested by Western blot using recombinant proteins and lysates from infected cells.

### Immunization of Mice

Four groups of 6 BALB/c mice (6-8 week-old females; Jackson Laboratories) were immunized 4 times at 3-week interval. Mice received injections of 20 μg of WNV-LPs into each quadriceps muscle in 100 μl of PBS, on the basis of the previously described immunization protocol for HCV-LPs (Qiao, M. et al. 2003 Hepatology 37:52-59). One group received prME-like particles (prME-LPs) alone; a second group received prME-LPs plus AS01B (50 μl); a third group received CprME-like particles (CprME-LPs) alone; and a final group received AS01B (50 μl) alone. The adjuvant AS01B, which contains monophosphoryl lipid A and QS21, was provided by GlaxoSmithiKline. Serum samples were collected before immunization and 2 weeks after each immunization and were analyzed for anti-M, -E, or -NS1 antibodies by both ELISA and virus neutralization assay.

### Induction of Anti-WNV Antibodies

Blood samples before immunization and 2 weeks after each immunization were collected from the tail vein and analyzed for Anti-E and -M antibodies by enzyme-linked immunosorbent assay (ELISA). Microtiter plate (96-well, Dynatech Lab) was coated with 100 μl of purified recombinant WNV E or M protein (2 μg/ml) in PBS and incubated at 4° C. overnight. Unbound protein was removed by washing wells with PBS/0.05% tween-20×4 times. The wells were blocked with 200 μl of 5% Nonfat Dry Milk (Carnation) in PBS/0.05% tween-20+4% normal goat serum (Sigma) and incubated at RT for 2-3 hours or 4° C. overnight. One hundred microliters of the testing serum diluted 1/100 in 5% milk in PBS/0.05% tween-20 was incubated at 37° C. for 1 h or 4° C. overnight. The wells were washed 6 times with PBS/0.05% tween-20 and 100 μl of 1/1000 HRP-conjugated goat anti-mouse IgG (Sigma) in 5% milk in PBS/0.05% tween-20 was added and incubated at 37° C. for 1 h. After washing wells 6 times with PBS+/0.05% tween-20, 100 μl ABTS (Kirkegaard & Perry Lab) was added and incubated until positive control reached OD 405 nm of 1.5. The OD of the negative control should not be over 0.05.

### Neutralization Assay

For the virus neutralization assay, serum samples were prepared in duplicate as serial two-fold dilutions in heat inactivated Dulbecco's modified minimal essential medium (DMEM) supplemented with 2% normal calf serum, 100 U/ml penicillin, 100 μg/ml streptomycin and dispensed at 50 μl/well into 96-well cell culture plates. Fifty microliters of DMEM containing 100 tissue culture infectious doses of WNV strain NY 1999 was added to each well and incubated at 37° C. for 60 min. Following neutralization, 100 μl of Vero cell suspension containing 10,000 cells was added to each well. The plates were incubated at 37° C./5% CO₂ and observed daily for 5 days for cytopathic effect and cell fusion. Inhibition titers were expressed as the reciprocal of the serum dilution that completely inhibited cytopathic effect in duplicate wells. WNV hyperimmune serum (ATCC) control (inhibition titer of 128) was used as positive control and normal mouse serum (inhibition titer <2) as negative control. Cells were fixed in 3.7% formaldehyde and stained with 40% methanol containing 0.1% crystal violet. Non-infected and mock-infected Vero cells served as negative controls.

### Animal Challenge

Mice were housed in biosafety level-3 conditions and were given food and water ad libitum. Mice were acclimatized for at least 1 week prior to challenge. Immunized mice and 6 age-matched, female BALB/c mice were inoculated intraperitoneally with 10⁴ pfu of WNV that had been derived from an infectious clone (Shi, P. Y. et al. 2002 J Virol 76:5847-5856). A group of 6 age-matched, female BALB/c mice were inoculated with diluent alone (PBS, 1% fetal bovine serum). Mice were weighed and scored daily for clinical signs of disease, including ruffled fur, hunching and paresis. Morbidity was defined as exhibition of >10% weight loss and/or clinical signs for ≧2 days. Mice that exhibited severe disease were killed. Surviving mice were killed 31 days after inoculation. Mice were bled on day 3 after inoculation. Spleens and brains were harvested from mice at death or on the day of killing, and blood was also harvested from mice that were killed. Brains were divided sagittally at the midline. One-half of each brain was processed for RNA extraction as described elsewhere (Kauffman, E. B. et al. 2003 J Clin Microbiol 41:3661-3667).

### WNV RNA Quantitation and Histology Assessment

The RNA from serum, spleens, and brains were analyzed for WNV in the envelope gene by real-time reverse-transcription-PCR (RT-PCR) with primers as described elsewhere (Kauffman, E. B. et al. 2003 J Clin Microbiol 41:3661-3667). RNA copies were calculated by use of a standard curve of 50 to 5×10⁵ copies of RNA per reaction and are reported as the number of copies per milliliter of serum or per gram of tissue. The thresholds of detection for serum, spleen and brain assays were 5×10³ copies/ml, 1.5× copies/g, and 7.5×10³ copies/g, respectively. Virus was titered using Vero cells (Kauffman, E. B et al. 2003 J Clin Microbiol 41:3661-3667). Fixed brains were sectioned, stained with hematoxylin/eosin (HE), and blindly assessed for abnormalities by light microscopy.

While the present invention has been described in some detail for purposes of clarity and understanding, one skilled in the art will appreciate that various changes in form and detail can be made without departing from the true scope of the invention. All figures, tables, and appendices, as well as patents, applications, and publications, referred to above, are hereby incorporated by reference.

1. A method for the production of virus-like particles (VLPs) from a West Nile Virus (WNV), said method comprising the steps of:
expressing a construct comprising the prM and E genes of a WNV, or a variant of the prM and/or E genes, in a baculoviral expression cassette and cloned under the control of a promoter in insect cells;
culturing the insect cells for a sufficient period of time to allow production of baculovirus particles; and
separating the VLPs from the baculoviral particles and the insect cells by lysis of the insect cells.

2. A method according to claim 1, wherein said insect cells are Sf9 cells or High5 cells.

3. A method according to claim 1, wherein said construct further comprises a region of nucleic acid encoding a signalase cleavage site in the prM gene to mediate the cleavage of prM to form the structural protein M.

4. A method according to claim 1, wherein said construct further comprises a region of nucleic acid encoding a furin cleavage site at the junction of the prM/E genes to mediate the cleavage of the polyprotein.

5. A method according to claim 1, wherein nucleotide sequence from a gene proximal to the prM and/or E genes in the sequence of the viral polyprotein is included in the construct.

6. A method according to claim 5, wherein said nucleotide sequence from the gene proximal to the prM and/or E genes is derived from the capsid (C) gene that abuts the prM gene, and/or the NS1 gene that abuts the E gene.

7. A method according to claim 1, wherein the strain of WNV is NY 99.

8. A preparation of WNV VLPs obtained by a method of claim 1.

9. A pharmaceutical composition comprising a preparation of WNV VLPs according to claim 8, optionally further comprising an adjuvant.

10. A method of diagnosis of a WNV-mediated disease, said method comprising the steps of:
contacting a VLP preparation according to claim 8 with a biological sample under conditions suitable for the formation of a polypeptide-antibody complex; and
detecting said complex.

11. (canceled)

12. A vaccine (immunogenic) composition comprising a preparation according to claim 8.

13. A method of vaccinating (immunizing) a subject against infection mediated by a WNV, comprising administering a vaccine (immunogenic) composition according to claim 12 to said subject.

14. (canceled)

15. A nucleotide construct comprising the prM and E genes of a WNV, or a variant of the prM and/or E genes, cloned under the control of a promoter in a baculoviral expression cassette, wherein said construct does not further comprise a region of nucleic acid encoding a furin cleavage site at the junction of the prM/E genes to mediate the cleavage of the polyprotein, and optionally wherein said construct further comprises a region of nucleic acid encoding a signalase cleavage site in the prM gene to mediate the cleavage of prM to form the structural protein M.

16. A nucleotide construct according to claim 15, wherein said promoter is a polyhedrin promoter or a p10 promoter.

17. A vector comprising a nucleotide construct according to claim 15.

18. An insect cell comprising a nucleotide construct according to claim 15.

19. A preparation comprising prME or ME WNV VLPs prepared by lysis of insect cells.

20. A pharmaceutical composition comprising a preparation of WNV VLPs according to claim 19 optionally further comprising an adjuvant.

21. A method of diagnosis of a WNV-mediated disease, said method comprising the steps of:
contacting a VLP preparation according to claim 19 with a biological sample under conditions suitable for the formation of a polypeptide-antibody complex; and
detecting said complex.

22. (canceled)

23. A vaccine (immunogenic) composition comprising a preparation according to claim 19.

24. A method of vaccinating (immunizing) a subject against infection mediated by a WNV, comprising administering a vaccine (immunogenic) composition according to claim 20 to said subject.

25. (canceled)


SEQUENCE LISTING (4 sequences)

SEQ ID NO: 1 - DNA, 19 bp, Artificial Sequence
  synthetic primer
ctatcaatcggcggagctc

SEQ ID NO: 2 - DNA, 18 bp, Artificial Sequence
  synthetic primer
acccagtgtcagcgtgca

SEQ ID NO: 3 - DNA, 51 bp, Artificial Sequence
  synthetic primer
gcgggatcctaatacgactcactatagggagtagttcgcctgtgtgagctg

SEQ ID NO: 4 - DNA, 20 bp, Artificial Sequence
  synthetic primer
gcttcccacatttgrtgytc